(19) United States
(12) Patent Application Publication
(10) Pub. No.: US 2013/0165689 A1
(43) Pub. Date: Jun. 27, 2013

(54) PROCESS FOR PREPARING ONE OR MORE COMPLEXING AGENTS SELECTED FROM METHYLGLYCINEDIACETIC ACID, GLUTAMIC ACID DIACETIC ACID AND SALTS THEREOF

(71) Applicants: Robert BAUMANN, Mannheim (DE); Markus Christian Biel, Mannheim (DE); Axel Franzke, Mannheim (DE); Alfred Oftring, Bad Duerkheim (DE); Friedhelm Teich, Edingen-Neckarhausen (DE); Paul Klingelhoefer, Mannheim (DE); Guido Henze, Tokyo (JP); Marie Katrin Schroeter, Dannstadt-Schauernheim (DE); Olesya Kister, Stuttgart (DE); Sabine Borchers, Erlenbach bei Kandel (DE); Pavel Tuzina, Mannheim (DE); Beate Deimling, Frankenthal (DE); Udo Leidel, Worms (DE)

(72) Inventors: Robert BAUMANN, Mannheim (DE); Markus Christian Biel, Mannheim (DE); Axel Franzke, Mannheim (DE); Alfred Oftring, Bad Duerkheim (DE); Friedhelm Teich, Edingen-Neckarhausen (DE); Paul Klingelhoefer, Mannheim (DE); Guido Henze, Tokyo (JP); Marie Katrin Schroeter, Dannstadt-Schauernheim (DE); Olesya Kister, Stuttgart (DE); Sabine Borchers, Erlenbach bei Kandel (DE); Pavel Tuzina, Mannheim (DE); Beate Deimling, Frankenthal (DE); Udo Leidel, Worms (DE)

(21) Appl. No.: 13/725,092

(22) Filed: Dec. 21, 2012

Related U.S. Application Data
(60) Provisional application No. 61/579,665, filed on Dec. 23, 2011.

(30) Foreign Application Priority Data
Apr. 17, 2012 (EP) 12164465.2

(51) Int. Cl. C07C 227/02

(52) U.S. Cl. CPC C07C 227/02
USPC 562/526; 502/345

(57) ABSTRACT

Process for preparing one or more complexing agents selected from methylglycinediacetic acid, glutamic acid diacetic acid and salts thereof

Process for preparing one or more complexing agents selected from methylglycinediacetic acid, glutamic acid diacetic acid and salts thereof by catalytic dehydrogenation of N,N-bis(2-hydroxyethyl)alanine and/or N,N-bis(2-hydroxyethyl)glutamic acid and/or salts thereof in the presence of alkali metal hydroxide, where a catalyst comprising copper and zirconium dioxide is used, the activation of which is a reduction, wherein the precursor of the catalyst in question has a degree of crystallization K, defined as

$$K = \frac{{I}_{K} \cdot 100}{{I}_{K} + {I}_{A}} ,$$

in the range from 0 to 50%.

The invention relates to a process for preparing one or more complexing agents selected from methylglycinediacetic acid, glutamic acid diacetic acid and salts thereof, starting from N,N-bis(2-hydroxyethyl)alanine and/or N,N-bis(2-hydroxyethyl)glutamic acid and/or salts thereof by catalytic dehydrogenation using alkali metal hydroxide.

Methylglycinediacetic acid (referred to hereinbelow in abbreviated form as MGDA) and glutamic acid diacetic acid (referred to hereinbelow as GLDA) or salts thereof are known complexing agents, particularly for use in detergents or dishwashing detergents. They are also used in powder or liquid detergent formulations for textile washing as builders and preservatives. In soaps, they prevent metal-catalyzed, oxidative decompositions, as also in pharmaceuticals, cosmetics and foods.

MGDA and GLDA and salts thereof can be prepared inter alia by catalytic dehydrogenation of N,N-bis(2-hydroxyethyl)alanine (referred to hereinbelow as ALDE) and N,N-bis(2-hydroxyethyl)glutamic acid (referred to hereinbelow in abbreviated form as GLDE) or of salts thereof in the presence of an alkali metal hydroxide.

The reaction of ALDE and GLDE or salts thereof can be represented by the following overall reaction equation:

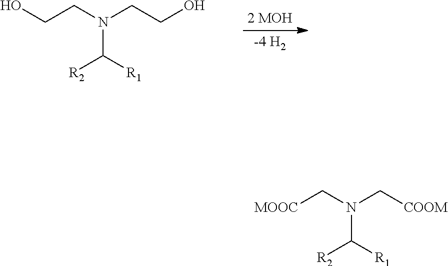

R₁ here is —COOX where X=alkali metal or hydrogen, R₂ is methyl in the case of ALDE or CH₂CH₂COOX where X=alkali metal or hydrogen in the case of GLDE. M is any desired alkali metal.

The reaction outlined above is a sequence of at least three reactions, which can be described as catalytic dehydrogenation with aldehyde formation, formation of the hydrate of the aldehyde and catalytic dehydrogenation of the hydrate of the aldehyde to the carboxylic acid. The overall sequence is referred to for the purposes of the present invention as “catalytic dehydrogenation”.

Preferably, R2 is methyl, i.e. N,N-bis(2-hydroxyethyl)alanine ALDE or salt thereof is reacted. Furthermore, M is preferably sodium, i.e. the reaction is carried out in the presence of sodium hydroxide solution.

The dehydrogenation of amino alcohols with alkali metal hydroxides using copper-based catalysts is described in detail in the prior art, for example in WO 2000/066539, EP 1 125 633, DE 69110447, JP 11158130, WO 2000/032310, WO 2003/033140, WO 2001/077054, PT 101870, PT 101452, WO 2003/051513, GB 2148287, WO 98/13140, JP 90037911 and EP 0 201 957.

Various copper-based catalysts have been used for the dehydrogenation of amino alcohols. As well as pure Raney copper (U.S. Pat. No. 4,782,183), which deactivates even after a short time as a result of sintering, variants of Raney copper doped with a very wide variety of metal ions have also been claimed (U.S. Pat. No. 5,292,936). For the purposes of increasing activity and stability, catalysts have also been described in the patent literature in which the active metal copper is anchored to an alkali-stable support. These include, for example, a system consisting of activated carbon and palladium (U.S. Pat. No. 5,627,125), but also carbon-free supports such as SiO₂, TiO₂ or ZrO₂ (U.S. Pat. No. 4,782,183 or WO 98/13140). In addition, nickel in the form of a sponge can also serve as support material, onto which a coating made of copper is applied (U.S. Pat. No. 7,329,778) which, in a further embodiment, is also admixed with iron in order to increase the selectivity of the dehydrogenation (WO 01/77054).

CN 101733100 describes a catalyst comprising copper and zirconium for the selective preparation of iminodiacetic acid by dehydrogenation of diethanolamine, the long-term activity of which can moreover be improved by means of a doping with further metal ions. The specified catalyst has amorphous fractions of copper and/or zirconium.

One problem in the preparation of methylglycinediacetic acid (MGDA) and glutamic acid diacetic acid (GLDA) or salts thereof from the corresponding dialkanolamines ALDE or GLDE or salts thereof is that, in the case of a procedure corresponding to the prior art at constantly high temperatures, by-products with lower effectiveness as complexing agents are formed to an increased degree. These include in particular compounds which originate from C—N or C—C bond breaks. In the case of the aminopolycarboxylate methylglycinediacetic acid trisodium salt (MGDA-Na₃), these are for example carboxymethylalanine disodium salt (C—N bond cleavage) and N-methyl-N-carboxymethylalanine (C—C bond cleavage).

It was therefore an object of the invention to provide a process, which is technically simple to carry out, for preparing MGDA and/or GLDA and/or salts thereof, according to which a product is obtained which has a high degree of purity without complex purification. Within the context of the present invention, a high degree of purity is synonymous with a high yield of at least 85 mol % relative to the desired product of value or, expressed in a different way, the by-products should constitute not more than 15% by weight, based on the desired product.

Accordingly, the process defined at the start has been found, also called process according to the invention for short. Furthermore, the catalyst defined at the start have been found. Furthermore, a process for producing catalysts has been found.

The attainment of the object consists in a process for preparing one or more complexing agents selected from methylglycinediacetic acid, glutamic acid diacetic acid and salts thereof by catalytic dehydrogenation of N,N-bis(2-hydroxyethyl)alanine and/or N,N-bis(2-hydroxyethyl)glutamic acid and/or salts thereof in the presence of an alkali metal hydroxide, where a catalyst comprising copper and zirconium dioxide is used, and where the activation of the catalyst is a reduction, wherein the non-activated precursor of the catalyst in question has a degree of crystallization K, defined as

$$K = \frac{{I}_{K} \cdot 100}{{I}_{K} + {I}_{A}} ,$$

in the range from 0 to 50%, preferably 0 to 30%, particularly preferably 1 to 30%, where the variables are defined as follows:

I_{K} is the integral over the intensity fractions L_{K} of the crystalline constituents of the precursor of the catalyst in question and

I_{A} is the integral over the intensity fractions L_{A} of the amorphous constituents of the precursor of the catalyst in question, in each case determined by X-ray diffractometry.

The degree of crystallization, K, describes the ratio of the intensity of the reflections of the crystalline constituents to the overall scattered intensity.

Without intending to give preference to a specific theory, it is assumed that the fraction of amorphous regions in the precursor of the catalyst corresponds essentially to the fraction of the amorphous regions of the active catalyst and accordingly the fraction of crystalline regions in the precursor of the catalyst corresponds essentially to the fraction of the crystalline regions of the active catalyst.

Preferably, the determination of the degree of crystallinity is carried out by X-ray diffractometry according to the method of intensity ratios with CuKa radiation in an angle range of the angle of diffraction 2θ of 5 to 80°. In this connection, it is possible to work with a step width 2θ of 0.02°, using an energy-dispersive X-ray detector or a X-ray detector with secondary-side monochromator, and also with primary-side and secondary-side variable diaphragm of size V20. Here, the intensity of the X radiation is measured as a function of the angle of diffraction 2θ. This intensity distribution is (least-squares-fit) adapted to the measured data according to the Pawley. The following factors are taken into account in this case: linear background, Lorentz and polarization correction, lattice parameters, space group, and crystallite size of the crystalline fractions (L_{X}). The intensity fractions L_{A} of the amorphous constituents of the non-activated precursor of the catalyst are fitted by four additional Lorentz functions with centers at 30.8° (2θ) 32.8° (2θ), 50° (2θ) and 59° (2θ) with adaptive amplitudes and half-widths.

N,N-Bis(2-hydroxyethyl)alanine and/or N,N-bis(2-hydroxyethyl)glutamic acid and salts thereof can be used in an enantiomerically pure form, for example as S enantiomer or as R enantiomer, or as racemate. In another variant, enantiomer mixtures can be used.

According to the invention, a catalyst comprising copper and zirconium dioxide is used, the non-activated precursor of which has a degree of crystallization in the range from 0 to 50%, preferably zero to 30%, particularly preferably 1 to 30, preferably determined on the precursor of the catalyst in question by X-ray diffractometry according to the method of intensity ratios, particularly preferably by measurement with a D8 Advance X-ray diffractometer from Bruker AXS GmbH, Karlsruhe, with CuKa radiation in an angle range 2θ from 5 to 80°, with a step width 2θ of 0.02°, using a Sol-X detector, using the modeling software TOPAS® from Bruker AXS GmbH, Karlsruhe to fit the peak profiles to the measured data and to determine the ratio of the intensity of the crystalline reflections to the intensity of the background which is attributed to the amorphous fraction. In this connection, zero % degree of crystallinity is to be understood as meaning that no measurable crystalline fractions can be ascertained by the method described above.

In one embodiment of the present invention, a catalyst comprising copper and zirconium dioxide is used which has a BET surface area of from 60 to 200 m²/g.

As regards the copper fraction, it is advantageous to use a catalyst comprising copper and zirconium dioxide which, after the reaction, comprises 1 to 50% by weight of copper, preferably 5 to 40% by weight, particularly preferably 10 to 30% by weight, based on the total weight of the catalyst.

Prior to the start of the process according to the invention or in situ during the process according to the invention, the non-activated precursor is activated, for example by reduction. Suitable reducing agents are, for example, magnesium, aluminum, zinc, alkali metals, or metal hydrides, for example lithium aluminum hydride, sodium borohydride, sodium hydride, also hydrazine. A particularly preferred reducing agent is hydrogen, pure or diluted with an inert gas, for example with noble gas or with nitrogen.

Catalyst—or its precursor— used in the process according to the invention can be used present in particulate form in a non-particulate form.

“Present in particulate form” is to be understood as meaning that the catalyst in question is present in the form of particles, the average diameter of which is in the range from 0.1 μm to 2 mm, preferably 0.001 to 1 mm, preferably in the range from 0.005 to 0.5 mm, in particular 0.01 to 0.25 mm.

“Present in non-particulate form” is to be understood as meaning that the catalyst, in at least one dimension (width, height, depth), has more than 2 mm, preferably at least 5 mm, where at least one other dimension, for example one or both other dimensions, can be less than 2 mm in size, for example in the range from 0.1 μm to 2 mm. In another variant, catalyst present in non-particulate form has three dimensions which have a measurement of more than 1 mm, preferably more than 2 mm, particularly preferably at least 3 mm, very particularly preferably at least 5 mm. A suitable upper limit is, for example, 10 m, preferably 10 cm.

Examples of catalysts which are present in non-particulate form are catalyst placed on metal meshes, for example steel meshes or nickel meshes, also wires such as steel wires or nickel wires, also shaped bodies, for example beads, Raschig rings, extrudates and tablets.

In one embodiment of the present invention, catalyst is used in the form of shaped bodies, for example in the form of tablets or extrudates.

Examples of particularly suitable dimensions of shaped bodies are tablets with measurements (radius-thickness 6.3 mm, 3.3 mm, 2.2 mm or 1.5-1.5 mm, and extrudates with a diameter in the range from 1.5 to 3 mm.

In one embodiment of the present invention, the process according to the invention is carried out at a temperature in the range from 160 to 210° C., preferably at a temperature in the range from 180 to 195° C.

In one embodiment of the present invention, the process according to the invention is carried out a pressure in the range from 5 to 100 bar absolute, preferably 8 to 20 bar absolute.

In one embodiment of the present invention, the process according to the invention is carried out with water as solvent, with or preferably without the use of organic solvent.

In one embodiment of the present invention, the process according to the invention is carried out such that a concentration of 1 to 50 g, preferably 10 to 50 g, of catalyst is selected per mole of ALDE or GLDE.

In one embodiment of the present invention, the process according to the invention is carried out such that, at the start of the reaction, a concentration of from 1 to 10 mol of ALDE or GLDE/l of water is selected, preferably 2 to 5 mol of ALDE or GLDE/l of water.

In one embodiment of the present invention, an excess of alkali metal hydroxide, based on ALDE or GLDE, is used. For example, it is possible to work with an excess in the range from 0.1 to 10 mol of alkali metal hydroxide, based on ALDE or GLDE, preferably 0.2 to 2 mol.

In one embodiment of the present invention, hydrogen formed during the process according to the invention is separated off in intervals or preferably continuously, for example via a pressure relief valve.

Optionally, MGDA, GLDA or salts thereof produced by the process according to the invention can also be after-treated. In the case of a suspension mode, the catalyst can be deactivated, sedimented and/or filtered off. In one embodiment, it is possible to carry out a bleaching e.g. with hydrogen peroxide or UV light.

Besides the salts of complexing agents themselves, i.e. aminopolycarboxylates, the corresponding aminocarboxylic acids MGDA and GLDA are also accessible by means of acidification.

A further aspect of the present invention is a catalyst, also called catalyst according to the invention for short, comprising copper and zirconium dioxide, wherein, prior to the activation, it has a degree of crystallization K, defined as

$$K = \frac{{I}_{K} \cdot 100}{{I}_{K} + {I}_{A}} ,$$

in the range from 0 to 50%, preferably 0 to 30%, particularly preferably 1 to 30%, where the variables are defined as follows:

I_{K} is the integral over the intensity fractions L_{K} of the crystalline constituents of the precursor of the catalyst and

I_{A} is the integral over the intensity fractions L_{A} of the amorphous constituents of the precursor of the catalyst, determined by X-ray diffractometry.

Catalyst—or its precursor— according to the invention can be present in particulate form or in non-particulate form.

“Present in particulate form” is to be understood as meaning that the catalyst in question is present in the form of particles, the average diameter of which is in the range from 0.1 μm to 2 mm, preferably 0.001 to 1 mm, preferably in the range from 0.005 to 0.5 mm, in particular 0.01 to 0.25 mm.

“Present in non-particulate form” is to be understood as meaning that the catalyst, in at least one dimension (width, height, depth), has more than 2 mm, preferably at least 5 mm, where at least one other dimension, for example one or both other dimensions, can be less than 2 mm in size, for example in the range from 0.1 μm to 2 mm. In another variant, catalyst present in non-particulate form has three dimensions which have one measurement of more than 2 mm, preferably at least 5 mm. A suitable upper limit is, for example, 10 m, preferably 10 cm.

Examples of catalysts which are present in non-particulate form are catalyst on metal meshes, for example steel meshes or nickel meshes, also on wires such as steel wires or nickel wires, also shaped bodies, for example beads, Raschig rings, extrudates and tablets.

In one embodiment of the present invention, catalyst is used in the form of shaped bodies, for example in the form of tablets or extrudates.

Examples of particularly suitable dimensions of shaped bodies are tablets with dimensions (radius-thickness) 6.3 mm, 3.3 mm, 2.2 mm or 1.5-1.5 mm, and extrudates with a diameter in the range from 1.5 to 3 mm.

A further aspect is a process for producing catalysts according to the invention and a process for producing precursors of catalysts according to the invention.

The process according to the invention for producing a catalyst comprises the following steps:
(a) provision of an acidic aqueous solution of at least one water-soluble copper salt and at least one water-soluble zirconium salt,
(b) precipitation of a precursor by increasing the pH, where the pH at the end of the precipitation is in the range from 8 to 14, preferably 10 to 12,
(c) reduction (activation) of the precursor.

Steps (a) to (c) are explained in more detail below.

The catalyst used in the present case is preferably produced by precipitation, starting from one or more water-soluble copper salts and one or more water-soluble zirconium salts and reduction of the precursor produced in this way. In one variant, the precursor can be washed or thermally treated before the reduction.

In this connection, water-soluble copper salts or zirconium salts should be understood as meaning those copper or zirconium compounds which have a solubility of at least 10 g/l at 25° C. in water or in aqueous mineral acid at a pH in the range from 1 to 5.

In one embodiment of the present invention, water-soluble copper salts are selected from nitrate, sulfate, oxalate, chloride, acetate and amine complexes of copper(II). Copper(II) nitrate is particularly preferably selected as water-soluble copper salt.

In one embodiment of the present invention, water-soluble zirconium salt is selected from nitrate, oxalate, chloride, sulfate and acetate of zirconium(IV), in neutral or in basic form, for example as oxychloride and oxynitrate. Preference is given to using zirconium oxychloride or zirconium oxynitrate as water-soluble zirconium salt.

In one embodiment of the present invention, in step (a), in the range from 10 to 500 g/l of water-soluble copper salt is dissolved in water or aqueous mineral acid.

In one embodiment of the present invention, in step (a), in the range from 10 to 650 g/l of water-soluble zirconium salt is dissolved in water or aqueous mineral acid.

In one embodiment of the present invention, in step (a), a solution is provided which comprises in total in the range from 10 to 650 g/l of water-soluble zirconium salt and in total in the range from 10 to 500 g/l of water-soluble copper salt.

Water-soluble copper salt and water-soluble zirconium salt can be dissolved separately or together in water.

Copper(II) and zirconium(IV) are present in water usually as aqua complexes which have a tendency towards protolysis, for example as hexaquocomplexes. For this reason, solution provided in step (a) can be acidic; it can for example have a pH in the range from 0.5 to 2.

The precipitation of the precursor, which for the purposes of the present invention can also be referred to as non-activated precursor, according to step (b) is achieved by increasing the pH of the acidic aqueous solution of at least one copper salt and at least one zirconium salt from step (a). At the end of the precipitation, the pH here is in the range from 8 to 14, preferably 10 to 12.

In one embodiment of the present invention, the pH during the precipitation reaction can be temporarily above 14 or below 8. In another embodiment of the present invention, the pH during the entire precipitation is in the range from 8 to 14.

The pH is preferably increased by mixing with at least one alkaline compound, preferably with alkali metal hydroxide, for example potassium hydroxide or with sodium hydroxide. Alkali metal hydroxide can be added in solid or in dissolved form, preference being given to adding alkali metal hydroxide in dissolved form.

In one embodiment of the present invention, step (b) is carried out at a temperature in the range from 5 to 50° C., preferably 20 to 30° C.

In one embodiment of the present invention, step (b) is carried out with stirring.

In one embodiment of the present invention, acidic aqueous solution of at least one copper salt and at least one zirconium salt on the one hand, and an aqueous solution of alkali metal hydroxide on the other hand, are simultaneously metered into a vessel, where precursor is precipitated out. In another embodiment of the present invention, acidic aqueous solution of at least one copper salt and at least one zirconium salt is introduced as initial charge and alkali metal hydroxide is metered in. In another embodiment of the present invention, aqueous solution of alkali metal hydroxide is introduced as initial charge and then the acidic aqueous solution of at least one copper salt and at least one zirconium salt is metered in. Here, the pH at the end of the precipitation of the precursor is set in the range from 8 to 12. Should the pH increase too much, then the pH can be reduced by adding mineral acid, the anhydride of which advantageously corresponds to the counterion of water-soluble copper(II) salt or water-soluble zirconium salt.

At the pH at the end of the precipitation, the mixture can be left to age, for example with stirring, for example over a period from 10 minutes to 3 hours.

After the precursor of the catalyst has precipitated out, precursor is separately off from the mother liquor, for example by filtration, sedimentation or centrifugation, preferably filtration. After the separation, purification operations can be carried out, for example washing.

In a preferred embodiment of the present invention, the precipitated precursor is washed with water.

In a preferred embodiment of the present invention, the washing is carried out to a residual conductivity of the filtrate of at most 1000 μS, particularly preferably to a residual conductivity of the filtrate of at most 500 μS.

In one embodiment of the present invention, step (b) can be followed by one or more thermal treatment steps, for example drying or calcination.

The drying is advantageously spray-drying or belt-drying. The drying of the precursor preferably takes place at temperatures in the range from 30 to 150° C.

If the precursor is to be calcined, then the calcination can be carried out at temperatures in the range from 150 to 800° C. Advantageously, however, the calcination should take place at temperatures in the range from 150° C. to 600° C.

Suitable devices for calcining the precursor are, for example, muffle furnaces, push-through furnaces and rotary-tube furnaces, also belt-calciners and belt-driers.

If the precursor is to be calcined, then a (average) residence time in the device provided for this purpose in the range from 10 minutes to 5 hours is possible.

In step (c), the precursor obtained as described above is reduced. The reduction can also be referred to as activation. The activation can be carried out for example with one or more reducing agents. Suitable reducing agents are for example hydrazine, metal such as zinc, magnesium, aluminum or alkali metals, also metal hydrides, in particular magnesium, aluminum, zinc, alkali metals, or metal hydrides, for example lithium aluminum hydride, sodium borohydride and sodium hydride. A particularly preferred reducing agent is hydrogen, pure or diluted with an inert gas, for example with noble gas or with nitrogen.

A suitable temperature for the reduction in step (c) is for example zero to 350° C., in the case of hydrogen preferably 150 to 260°.

The present invention further provides a process for producing precursors of catalysts according to the invention. The process according to the invention for producing precursors of catalysts according to the invention comprises the steps (a) and (b) of the process according to the invention for producing catalysts according to the invention and optionally washing and/or thermal treatment, but no activation according to step (c). The present invention further provides precursors for catalysts according to the invention.

The invention is described in more detail below by reference to working examples.

The degree of crystallization of the non-activated precursor of the catalyst was determined by the method of intensity ratios (cf. F. H. Chung and D. K. Smith: “Industrial Application of X-Ray Diffraction”, M. Dekker, 2000, pp. 496-499). Measurement is advantageously carried out on a D8 Advance diffractometer from Bruker AXS GmbH, Karlsruhe (CuKa radiation, Bragg-Brentano Geometry, Sol-X detector, 5-80° (20), step width 0.02° (20) with variable V20 diaphragm primary-side and secondary-side). In a modeling software (TOPAS® Bruker AXS GmbH, Karlsruhe), the peak profiles were fitted to the measured data and the ratio was determined.

The two crystalline fractions were described by reference to their lattice parameters. CuO: C2/c, a=4.6 Å, b=3.4 Å, c=5.3 Å, b=99.2° ZrO₂: P42/nmc, a=3.6 Å, c=5.2 Å. The amorphous background was modeled with individual broad peaks at 30.8° C. (2θ), 32.8° (2θ), 50° (2θ) and 59° (2θ).

I. Preparation of catalysts according to the invention and of comparative catalysts

I.1 Preparation of catalyst K.1 according to the invention Composition before the reduction: 77.5% by weight ZrO₂: 22.5% by weight CuO.

414 g of zirconium oxynitrate and 123 g of copper nitrate were dissolved in 3750 ml of water at room temperature in a stirred flask fitted with stirrer, heating jacket, pH electrode and thermometer. The pH of the solution obtained in this way was just below 1. Stirring was carried out with 170 revolutions per minute (rpm) and 25% by weight of sodium hydroxide solution aqueous at room temperature was added over a period of 10 minutes. A suspension was formed. The end of the precipitation was reached when the pH of the suspension was 10.5. After the end of the precipitation, the suspension was after-stirred for a further period of 15 minutes at room temperature. The pH was maintained at 10.5 during this time by adding dilute nitric acid. The suspension was then filtered undiluted through a suction filter and the filter cake was washed with water. The moist filter cake was dried at 105° C. for 16 hours and then calcined for 3 hours at 490° C. under an air atmosphere. The degree of crystallization was determined on the precursor VS.1 obtained in this way; see table 1.

Precursor VS.1 was reduced in a nitrogen-hydrogen stream at 230° C. over the course of 3 hours. While introducing a nitrogen stream (room temperature), the mixture was left to cool to room temperature. This gave catalyst K.1 according to the invention. Catalyst K.1 according to the invention was removed under nitrogen, drawn off in a glove box with nitrogen atmosphere and transferred with demineralized water through which nitrogen had been blown beforehand.

I.2 Preparation of the catalyst K.2 according to the invention Composition before reduction: 80% by weight ZrO₂: 20% by weight CuO

382 g of zirconium oxychloride and 109.3 g of copper nitrate were dissolved in 3750 ml of water at room temperature in a stirred flask fitted with stirrer, heating jacket, pH electrode and thermometer. The pH of the solution obtained in this way was just below 1. Stirring was carried out at 170 revolutions per minute (rpm) and 25% by weight of sodium hydroxide solution aqueous at room temperature were added over a period of 10 minutes. A suspension was formed. The end of the precipitation was reached when the pH of the suspension was 10.5. When the precipitation was complete, the suspension was after-stirred for a further period of 15 minutes at room temperature. The pH was held at 10.5 during this time by adding dilute hydrochloric acid. The suspension was then filtered undiluted through a suction filter and the filter cake was washed with water. The moist filter cake was dried for 16 hours at 105° C. and then calcined for 3 hours at 490° C. under an air atmosphere. The degree of crystallization was determined on the precursor VS.2 obtained in this way; see table 1.

Precursor VS.2 was reduced in a nitrogen-hydrogen stream at 230° C. over the course of 3 hours. While introducing a nitrogen stream (room temperature), the mixture was left to cool to room temperature. This gave catalyst K.2 according to the invention. Catalyst K.2 according to the invention was removed under nitrogen, drawn off in a glove box with nitrogen atmosphere and transferred with dermineralized water through which nitrogen had been blown beforehand.

I.3 Preparation of the catalyst K.3 according to the invention Composition before reduction: 88% by weight ZrO₂: 12% by weight CuO

411 g of zirconium oxychloride and 66.5 g of copper nitrate were dissolved in 3750 ml of water at room temperature in a stirred flask fitted with stirrer, heating jacket, pH electrode and thermometer. The pH of the solution obtained in this way was just below 1. Stirring was carried out at 170 revolutions per minute (rpm) and 25% by weight of sodium hydroxide solution aqueous at room temperature were added over a period of 10 minutes. A suspension was formed. The end of the precipitation was reached when the pH of the suspension was 10.5. When the precipitation was complete, the suspension was after-stirred for a further period of 15 minutes at room temperature. The pH was kept at 10.5 during this time by adding dilute hydrochloric acid. The suspension was then filtered undiluted through a suction filter and the filter cake was washed with water. The moist filter cake was dried for 16 hours at 105° C. and then calcined for 3 hours at 550° C. under an air atmosphere. The degree of crystallization was determined on the precursor VS.3 obtained in this way; see table 1.

Precursor VS.3 was reduced in a nitrogen-hydrogen stream at 230° C. over the course of 3 hours. While introducing a nitrogen stream (room temperature), the mixture was left to cool to room temperature. This gave catalyst K.3 according to the invention. Catalyst K.3 according to the invention was removed under nitrogen, drawn off in a glove box with nitrogen atmosphere and transferred with demineralized water through which nitrogen had been blown beforehand.

I.4 Preparation of the comparison catalyst V-K.4 Composition before reduction: 77.5% by weight ZrO₂: 22.5% by weight CuO.

370 g of zirconium oxychloride and 123 g of copper nitrate were dissolved in 3750 ml of water at room temperature in a stirred flask fitted with stirrer, heating jacket, pH probe and thermometer. The pH of the solution obtained in this way was just below 1. Stirring was carried out at 170 revolutions per minute (rpm) and 25% by weight of sodium hydroxide solution aqueous at room temperature were added over a period of 10 minutes. A suspension was formed. The end of the precipitation was reached when the pH of the suspension was 10.5. The suspension was after-stirred at room temperature over a period of 15 minutes. The pH was kept at 10.5 during this time by adding dilute hydrochloric acid. The pH of the suspension was subsequently reduced to 7 by adding hydrochloric acid. Then, the suspension was filtered undiluted through a suction filter and the filter cake was washed with water. The moist filter cake was dried at 105° C. for 16 hours and then calcined for 3 hours at 490° C. under an air atmosphere. The degree of crystallization was determined on the comparison precursor V-VS.4 obtained in this way; see table 1.

Comparison precursor V-VS.4 was reduced in a nitrogen-hydrogen stream at 230° C. over the course of 3 hours. While introducing a nitrogen stream (room temperature), the mixture was left to cool to room temperature. This gave comparison catalyst V-K.4. Comparison catalyst V-K.4 was removed under nitrogen, drawn off in a glove box with nitrogen atmosphere and transferred with demineralized water through which nitrogen had been blown beforehand.

I. Preparation of MGDA with the help of catalysts according to the invention and with comparison catalysts

II.1 Preparation of an aqueous N,N-bis(2-hydroxyethyl)alanine sodium salt solution

4.365 kg (49.00 mol) of L-alanine were suspended in 2.623 kg of water and admixed with 3.897 kg (49.00 mol) of 50.3% by weight of sodium hydroxide solution. The resulting solution was poured into a 20 liter autoclave (material 2.4610) and, after being rendered inert, supplied with 20 bar of nitrogen. 4.749 kg (107.8 mol) of ethylene oxide were then metered in at 40 to 45° C. over the course of 12.5 h and the mixture was after-stirred for 3 hours at this temperature. After removing the unreacted residues of ethylene oxide, the autoclave was emptied. This gave 15.634 kg of aqueous reaction product (N,N-bis(2-hydroxyethyl)alanine sodium salt solution) in the form of a clear, colorless, viscous solution.

II.2 Oxidative dehydrogenation, general procedure

279.5 g (0.99 mol based on alanine) of the above aqueous N,N-bis(2-hydroxyethyl)alanine starting solution were introduced as initial charge with 184 g (2.29 mol) of 50% by weight sodium hydroxide solution, 32 g of water and 30 g of the respective catalyst according to the invention or of the comparison catalyst in a 1.7 liter autoclave (material 2.4610). The autoclave was closed, supplied with 5 bar of nitrogen and then heated to 190° C. over the course of 2 hours. The mixture was stirred at 190° C. over a period of 16 hours at 500 rpm. The resulting hydrogen was drawn off continuously via a pressure relief valve regulating at 10 bar. Cooling to room temperature and decompression were then followed by flushing the autoclave at room temperature with nitrogen and diluting the reaction product with 400 g of water. This gave a clear, colorless, viscous solution which comprised primarily MGDA-Na₃. The yield (=selectivity-conversion) of methylglycine-N,N-diacetic acid trisodium salt (MGDA-Na₃), based on alanine used, and also the yield of carboxymethylalanine disodium salt (CMA-Na2), likewise based on alanine used, were determined by means of HPLC.

TABLE 1
Composition of catalysts according to the
invention and their use for preparing MGDA

| | | Degree of | | | |
|---|---|---|---|---|---|
| | Cu content | crystal- | Yield of | Yield of | CMA-Na₂/ |
| | [% by | lization | MGDA- | CMA-Na₂ | MGDA-Na₃ |
| Catalyst | weight] | [%] | Na₃ [%] | [%] | ratio |
| K.1 | 18.8 | 10 | 93.3 | 3.7 | 0.040 |
| K.2 | 16.7 | 22 | 88.3 | 5.3 | 0.060 |
| K.3 | 9.6 | 42 | 86.7 | 10.3 | 0.119 |
| V-K.4 | 18.8 | 55 | 82.7 | 16.8 | 0.203 |

Degree of crystallization indicates the degree of crystallization of the non-activated precursor of the catalyst which has been determined as described above.

The examples show that low ratios of the undesired cleavage product CMA-Na₂ to the product of value MGDA-Na₃ are obtained when the degree of crystallization of the non-activated precursor of the catalyst is in the range from 0 to 50%.

By contrast, in the comparative example, the ratio of CMA-Na₂ to MGDA-Na₃ is more unfavorable and the yield of MGDA-Na₃ is lower.

We claim:

1. A process for preparing one or more complexing agents selected from methylglycine diacetic acid, glutamic acid diacetic acid and salts thereof by catalytic dehydrogenation of N,N-bis(2-hydroxyethyl)alanine and/or N,N-bis(2-hydroxyethyl) glutamic acid and/or salts thereof in the presence of alkali metal hydroxide, where a catalyst comprising copper and zirconium dioxide is used, the activation of which is a reduction, wherein the non-activated precursor of the catalyst in question has a degree of crystallization K, defined as
$$K = \frac{{I}_{K} \cdot 100}{{I}_{K} + {I}_{A}} ,$$
in the range from 0 to 50%, where the variables are defined as follows:
I_{K} is the integral over the intensity fractions L_{K} of the crystalline constituents of the precursor of the catalyst in question and
I_{A} is the integral over the intensity fractions L_{A} of the amorphous constituents of the precursor of the catalyst in question, in each case determined by X-ray diffractometry.

2. The process according to claim 1, wherein the non-activated precursor of the catalyst in question has a degree of crystallization K in the range from 0 to 30%.

3. The process according to claim 1 or 2, wherein the activated catalyst comprises 1 to 50% by weight of copper, based on the total weight of the catalyst.

4. The process according to claim 3, wherein the activated catalyst comprises 5 to 40% by weight of copper, based on the total weight of the catalyst.

5. The process according to claim 4, wherein the activated catalyst comprises 10 to 30% by weight of copper, based on the total weight of the catalyst.

6. The process according to any one of claims 1 to 5, wherein the complexing agent is methylglycinediacetate.

7. The process according to any one of claims 1 to 6, wherein the alkali metal hydroxide selected is sodium hydroxide.

8. The process according to any one of claims 1 to 7, wherein the precursor of the catalyst is prepared by precipitation, starting from one or more water-soluble copper salts and one or more water-soluble zirconium salts.

9. The process according to claim 8, wherein the pH at the end of the precipitation of the precursor of the catalyst is in the range from 8 to 14.

10. A catalyst comprising copper and zirconium dioxide, wherein, before the activation, it has a degree of crystallization K, defined as
$$K = \frac{{I}_{K} \cdot 100}{{I}_{K} + {I}_{A}} ,$$
in the range from 0 to 50%, where the variables are defined as follows:
I_{K} is the integral over the intensity fractions L_{K} of the crystalline constituents of the precursor of the catalyst and
I_{A} is the integral over the intensity fractions L_{A} of the amorphous constituents of the precursor of the catalyst, in each case determined by X-ray diffractometry.

11. A process for producing a catalyst, comprising the following steps
(a) provision of an acidic aqueous solution of at least one copper salt and at least one zirconium salt,
(b) precipitation of a precursor by increasing the pH, where the pH at the end of the precipitation is in the range from 8 to 12,
(c) reduction of the precursor.

12. The process according to claim 11, wherein the precursor after step (b) has a degree of crystallization K, defined as
$$K = \frac{{I}_{K} \cdot 100}{{I}_{K} + {I}_{A}} ,$$
in the range from 0 to 50%, where the variables are defined as follows:
I_{K} is the integral over the intensity fractions L_{K} of the crystalline constituents of the precursor of the catalyst and
I_{A} is the integral over the intensity fractions L_{A} of the amorphous constituents of the precursor of the catalyst, in each case determined by X-ray diffractometry.

13. A precursor of a catalyst according to claim 10, wherein it has a degree of crystallization K, defined as
$$K = \frac{{I}_{K} \cdot 100}{{I}_{K} + {I}_{A}} ,$$
in the range from 0 to 50%, where the variables are defined as follows:
I_{K} is the integral over the intensity fractions L_{K} of the crystalline constituents of the precursor of the catalyst and
I_{A} is the integral over the intensity fractions L_{A} of the amorphous constituents of the precursor of the catalyst, in each case determined by X-ray diffractometry.

14. A process for producing a precursor of a catalyst according to claim 10 or 13, comprising the following steps:
(a) provision of an acidic aqueous solution of at least one copper salt and at least one zirconium salt,
(b) precipitation of a precursor by increasing the pH, where the pH at the end of the precipitation is in the range from 8 to 12.
